(19) United States
(12) Patent Application Publication
(10) Pub. No.: US 2013/0190602 A1
(43) Pub. Date: Jul. 25, 2013

(54) 2D3D REGISTRATION FOR MR-X RAY FUSION UTILIZING ONE ACQUISITION OF MR DATA

(71) Applicants: Rui Liao, Princeton Junction, NJ (US); James G. Reisman, Princeton, NJ (US); Christophe Chefd'hotel, Jersey City, NJ (US); Steven Michael Shea, Baltimore, MD (US)

(72) Inventors: Rui Liao, Princeton Junction, NJ (US); James G. Reisman, Princeton, NJ (US); Christophe Chefd'hotel, Jersey City, NJ (US); Steven Michael Shea, Baltimore, MD (US)

(73) Assignee: Siemens Corporation, Iselin, NJ (US)

(21) Appl. No.: 13/353,633

(22) Filed: Jan. 19, 2012

(51) Int. Cl. A61B 5/055; A61B 6/00

(52) U.S. Cl. 600/411

(57) ABSTRACT

Systems and methods for 2D3D registration of apply MR volumes and X-ray images using DRR techniques. A bone classifier is trained from co-registered UTE1, UTE2 and CT prior images. Dual-echo MR UTE1 and UTE2 images are acquired from a patient. The bone structure of the patient is classified and a labeled segmentation is generated. A DRR image is generated from the labeled segmentation and is registered with an X-ray image of the patient. The registration methods are implemented on a processor based system.

### BACKGROUND OF THE INVENTION

The present invention relates generally to medical imaging and more particularly to 2D3D registration of MR volumes with X-ray images.

2D X-ray fluoroscopy has been a preferred modality routinely used for interventional and hybrid medical procedures. It can provide real-time monitoring of the procedure and visualization of the device location. However, anatomic structures are typically not delineated by fluoroscopy because soft tissues are not distinguishable by X-rays. In order to augment the view of the anatomies and help the doctor navigate the device to the target area, pre-operative high quality computed tomography (CT) and/or magnetic resonance (MR) volumes can be fused with the intra-operative fluoroscopic images, for which 2D3D registration of the coordinate systems of the two modalities is needed.

One technique for 2D3D registration between CT volumes and X-ray images is based on digitally reconstructed radiographs (DRRs), which simulate the X-ray image by ray-casting through the CT volume. The generated DRRs are very close to the real X-ray projections due to the underlying similar physics for CT and X-ray imaging. A DRR-based method for registering MR volume is much more difficult, because the physics for MR and X-ray imaging is completely different.

Rapid and high quality 2D3D registration of MR volumes and X-ray images based on DRRs is believed currently not to be available.

Accordingly, improved and novel systems and methods for 2D3D registration of MR volumes and X-ray images using DRR techniques are required.

### BRIEF SUMMARY OF THE INVENTION

Aspects of the present invention provide systems and methods to register an X-ray image of a patient with a DRR generated from an MR volume containing an UTE1 and a UTE2 volume to align the X-ray image with the MR image of the patient.

In accordance with an aspect of the present invention, a method is provided for aligning a two-dimensional (2D) X-ray image of a patient with a Magnetic Resonance (MR) volume, comprising: creating data representing a bony structure classifier from three-dimensional (3D) image data generated from a plurality of individuals, acquiring with a Magnetic Resonance Imaging (MRI) device from the patient a dual echo signal volume containing an ultra-short echo time (UE1) volume and a standard echo time (UTE2) volume, a processor generating a labeled segmentation of the bony structure of the patient by using data representing the UTE1 and UTE2 volumes and the bony structure classifier, the processor generating a digitally reconstructed radiograph (DRR) image from the labeled segmentation of the bony structure and the processor registering the DRR image with the 2D X-ray image of the patient.

In accordance with a further aspect of the present invention, the method is provided, wherein the MR volume of the patient is aligned with the 2D X-ray image.

In accordance with yet a further aspect of the present invention, the method is provided, wherein the DRR image is generated by the processor from the labeled segmentation by using corresponding Hounsfield Units.

In accordance with yet a further aspect of the present invention, the method is provided, wherein the DRR is generated by using ray-casting through the acquired MR volume.

In accordance with yet a further aspect of the present invention, the method is provided, wherein the DRR is generated by using GPU-based acceleration.

In accordance with yet a further aspect of the present invention, the method is provided, wherein the DRR is generated by using ray-casting through the acquired MR volume and GPU-based acceleration.

In accordance with yet a further aspect of the present invention, the method is provided, wherein the bony structure is cortical bone.

In accordance with yet a further aspect of the present invention, the method is provided, further comprising: the processor generating a mesh of mesh triangles representing the labeled segmentation, the processor calculating an intersection of a ray and a mesh triangle and the processor calculating a distance between an in intersection and an out intersection of the ray.

In accordance with yet a further aspect of the present invention, the method is provided, wherein the labeled segmentation includes a label air, a label fat or soft tissue and a label bone.

In accordance with yet a further aspect of the present invention, the method is provided, wherein atlas information is incorporated into the bony structure classifier.

In accordance with another aspect of the present invention, a system is provided to align a two-dimensional (2D) X-ray image of a patient with a Magnetic Resonance (MR) volume, comprising: a memory enabled to store data, a processor enabled to execute instructions to perform the steps receiving data representing a bony structure classifier from three-dimensional (3D) image data generated from a plurality of patients, receiving data acquired with a Magnetic Resonance Imaging (MRI) device from the patient representing a dual echo signal volume containing an ultra-short echo time (UTE1) volume and a standard echo time (UTE2) volume, generating a labeled segmentation of the bony structure of the patient by using data representing the UTE1 and UTE2 volumes and the bony structure classifier, generating a digitally reconstructed radiograph (DRR) image from the labeled segmentation of the bony structure and registering the DRR image with the 2D X-ray image of the patient.

In accordance with yet another aspect of the present invention, the system is provided, wherein the MR volume of the patient is aligned with the 2D X-ray image.

In accordance with yet another aspect of the present invention, the system is provided, wherein the DRR image is generated by the processor from the labeled segmentation by using corresponding Hounsfield Units.

In accordance with yet another aspect of the present invention, the system is provided, wherein the DRR is generated by using ray-casting through the acquired MR volume.

In accordance with yet another aspect of the present invention, the system is provided, wherein the DRR is generated by using GPU-based acceleration.

In accordance with yet another aspect of the present invention, the system is provided, wherein the DRR is generated by using ray-casting through the acquired MR volume and GPU-based acceleration.

In accordance with yet another aspect of the present invention, the system is provided, wherein the bony structure is cortical bone.

In accordance with yet another aspect of the present invention, the system is provided, further comprising generating a mesh of mesh triangles representing the labeled segmentation, calculating an intersection of a ray and a mesh triangle and calculating a distance between an in intersection and an out intersection of the ray.

In accordance with yet another aspect of the present invention, the system is provided, wherein the labeled segmentation includes a label air, a label fat or soft tissue and a label bone.

In accordance with yet another aspect of the present invention, the system is provided, wherein atlas information is incorporated into the bony structure classifier.

### BRIEF DESCRIPTION OF THE DRAWINGS

FIG. 1 illustrates an UTE2 image;

FIG. 2 illustrates an UTE1 image;

FIG. 3 illustrates various steps of a method in accordance with one or more aspects of the present invention;

FIG. 4 illustrates a standard CT image;

FIG. 5 a pseudo-CT image from UTE1 and UTE2 acquisitions in accordance with various aspects of the present invention;

FIG. 6 illustrates images from the same object created with different methods;

FIG. 7 illustrates steps performed in accordance with various aspects of the present invention; and

FIG. 8 illustrates a system enabled to perform steps of methods provided in accordance with various aspects of the present invention.

### DETAILED DESCRIPTION

It is known that a DRR-based method for registering an MR volume is much more difficult than registering a CT volume, because the physics for MR and X-ray imaging is completely different. For example, the bony structure is usually not picked up well by MR using the standard protocol and can be confused with air or soft tissues. In particular, what is typically seen on MRI is the bone marrow or phrased in another way: the fat mixed into a spongy matrix. The outer/hard bone shell (cortical bone) surrounding the matrix is not seen with standard MR because there simply is no signal. For registration purpose, the diminished bony structures in MR volume do not correspond well to the highly opaque bony structures showed in the X-ray image, which can be misleading and lead to wrong registration.

As an aspect of the present invention a 2D3D registration technique for aligning MR volumes with X-ray images is provided by generating DRRs using one specialized MR acquisition, named ultra-short echo time (UTE) MR imaging. One aspect of UTE imaging is acquisition of an image at an “ultra-short” echo time on the range of 50-100 microseconds, which is roughly 10 to 20 times shorter than the shortest TE's (echo time) acquired with standard MR imaging methods. As such, the resulting images capture cortical bone and other very short T2 species, which is not present in standard images. This is described in “[7]. Robson et al., Clinical ultrashort echo time imaging of bone and other connective tissues, NMR Biomed. 2006: 19:765-780” which is incorporated herein by reference.

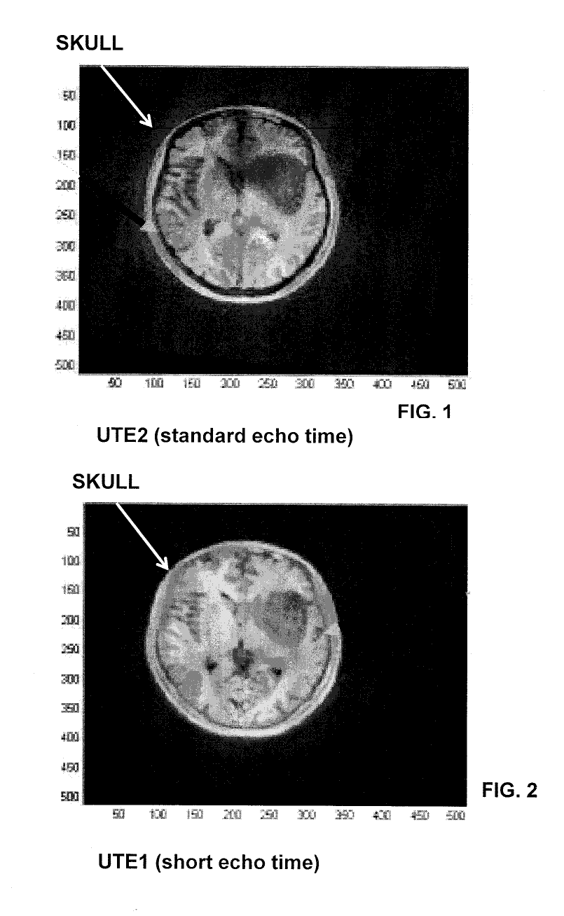

The UTE technique can produce multiple MR images with different contrasts as opposed to serially acquiring three or more acquisitions in the more standard approach. In addition, depending on the settings on the echo time there can be variability of responses among the multiple MR images. Compared to the UTE scan with a standard echo time (UTE2) as illustrated in FIG. 1, the UTE scan with an extra or ultra short echo time (UTE1) responds to the bony structure more strongly with a higher intensity value as illustrated in FIG. 2.

In accordance with an aspect of the present invention a 2D3D registration technique for aligning MR volumes with X-ray images is provided by generating DRRs using one specialized MR acquisition, named ultra-short echo time (UTE) MR imaging and as described in “[6] Bergin C J, Pauly J M, Macovski A, “Lung parenchyma: projection reconstruction MR imaging”, Radiology. 1991 June; 178(2):777-81.”

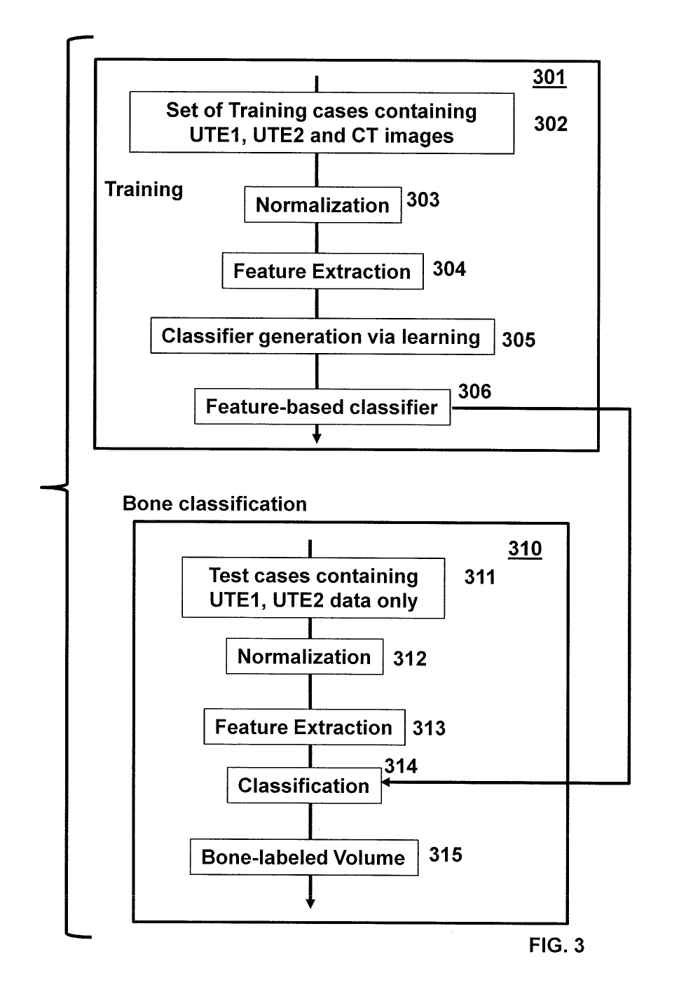

The UTE technique can produce multiple MR images with different contrasts as opposed to serially acquiring three or more acquisitions in the more standard approach. In addition, depending on the settings on the echo time there can be variability of responses among the multiple MR images. Compared to the UTE scan with a standard echo time (UTE2) as illustrated in FIG. 1, the UTE scan with an extra short or ultra-short echo time (UTE1) responds to the bony structure more strongly with a higher intensity value as illustrated in FIG. 2. Therefore, a bone classifier can be trained from the co-registered UTE1, UTE2 and CT volumes and the MR volume is then labeled (segmented) by the trained classifier into three segments: air, fat/soft tissue and bone as illustrated in FIG. 3.

The method as provided in accordance with an aspect of the present invention contains two phases which are each performed by a computing device with a processor: a training phase 301 and a bone classification phase 310. In the training phase, a set of training images containing UTE1, UTE2 and CT images are provided to a processor which first performs a normalization step 303, followed by a feature extraction step 304. The processor generates a classifier for a bone containing feature via a learning step 305 and makes the feature based classifier available in step 306.

Classifiers are known. A classifier is described in “[5] Y. Freund and R. E. Schapire, A decision-theoretic generalization of on-line learning and an application to boosting. J. Comput. Syst. Sci., 55(1):119-139, 1997,” which is incorporated herein by reference.

In a separate but related classification step 310, the processor is provided with UTE1 and UTE2 image data, but no CT images in a step 311, followed by a normalization step 312 and feature extraction step 313. Classification of the extracted features of step 313 is performed by using the classifier of step 306. The labeled or segmented image based on the classifier is provided in step 315.

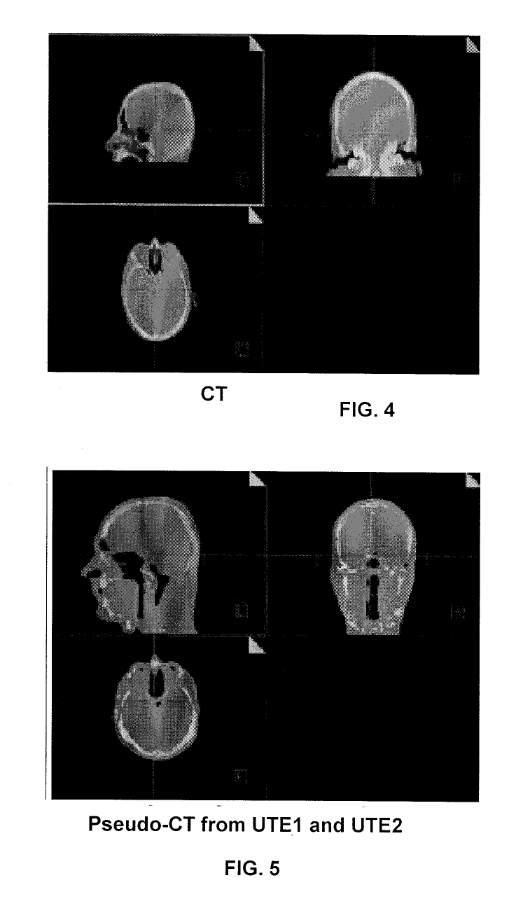

DRRs then are generated from the labeled segmentation using the corresponding Hounsfield Units (HUs), which correspond much more closely to the real X-ray projections than the DRRs generated from the original MR volume. This is illustrated in FIGS. 4 and 5 wherein FIG. 4 shows a CT image and FIG. 5 are HUs generated from UTE1 and UTE2 volumes.

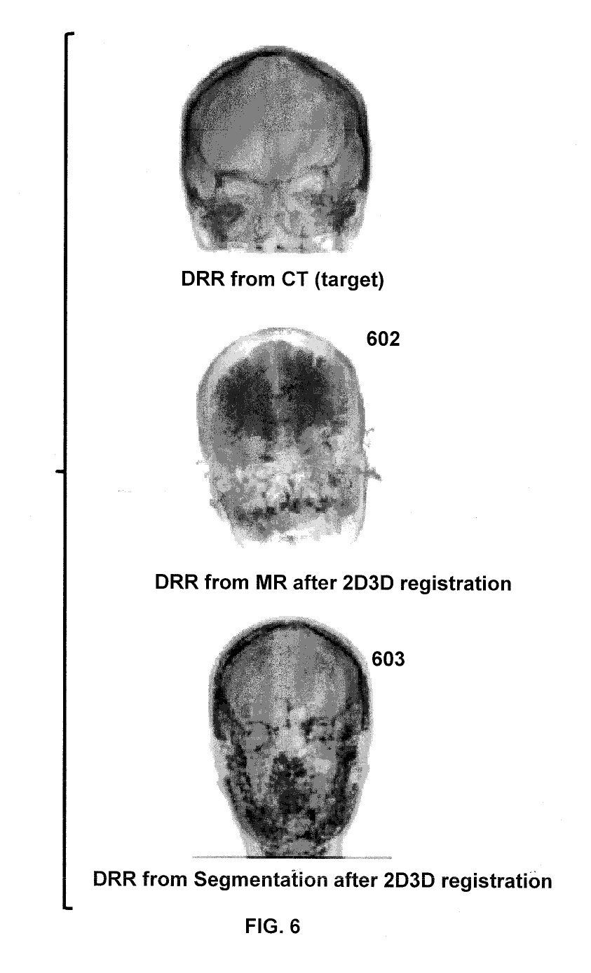

2D3D registration which utilizes the native X-ray images (versus digitally subtracted angiography showing the vessels) is largely driven by highly opaque objects, i.e. the bony structures. DRR-based registration utilizing the labeled segmentation with the corresponding HUs tends to provide much more accurate and robust performance compared to the case using the original MR volume. This is illustrated in FIG. 6.

FIG. 6 illustrates 2D3D registration using DRRs from labeled segmentation (603) with the corresponding HUs resulting in a correct alignment to the target (i.e. DRR from the ground-truth CT volume 601), while 2D3D registration using DRRs from the original MR volume 602 results in a wrong alignment of the scalp to the skull, due to the diminishing of the skull in the MR volume.

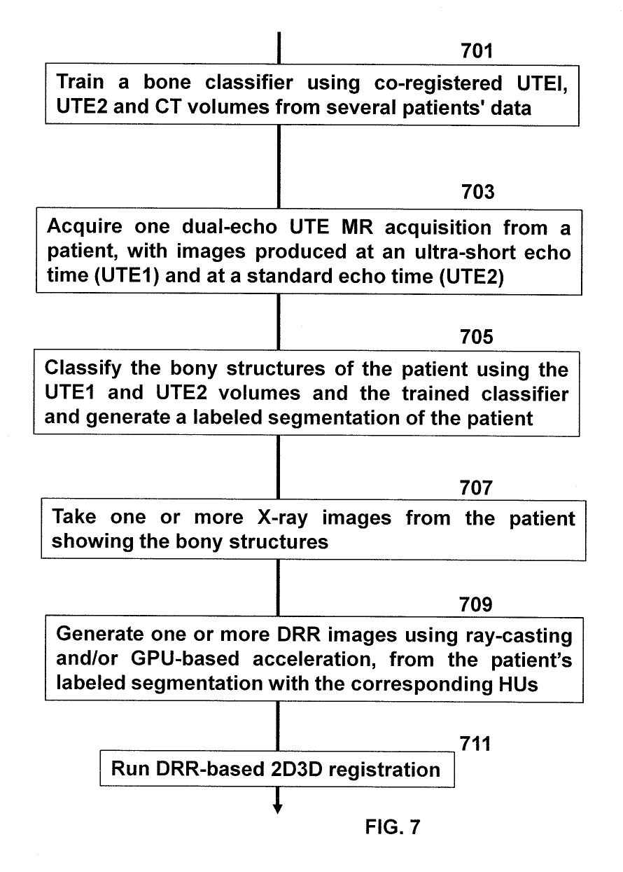

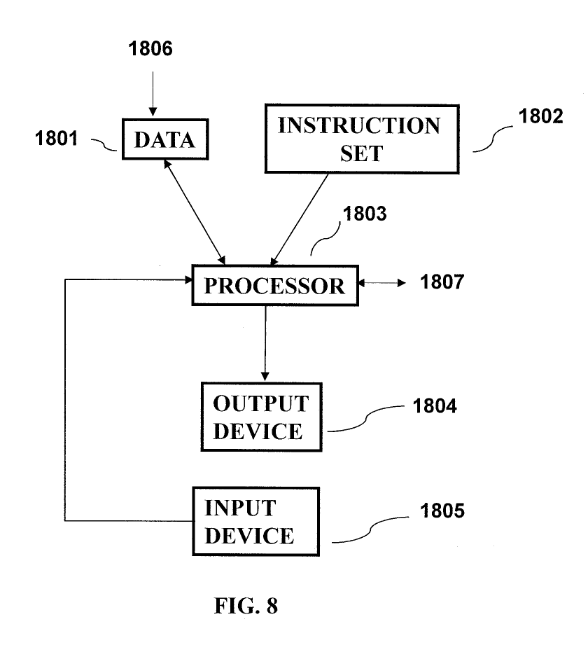

A method for 2D3D image registration that provided herein in accordance with various aspects of the present invention comprises the following steps, which are illustrated in FIG. 7:

1) Train a bone classifier using co-registered UTE1, UTE2 and CT volumes from several patients' data, as provided herein above and illustrated in FIG. 7 (step 701);
2) For a new case, one dual-echo U1E MR acquisition is acquired from a patient, with images produced at an ultra-short echo time (UTE1) and at a standard echo time (UTE2) (step 703);
3) Classify the bony structures of the patient using the UTE1 and UTE2 volumes and the trained classifier and generate a labeled segmentation of the patient as provided herein above and illustrated in FIGS. 3, 4 and 5 (step 705);
4) Take one or more X-ray images from the patient showing the bony structures, for 2D3D registration purpose (step 707);
5) Generate one or more DRR images using ray-casting and/or GPU-based acceleration, from the patient's labeled segmentation with the corresponding HUs, for 2D3D registration purpose (step 709); and
6) Run DRR-based 2D3D registration (step 711).

The herein provided 2D3D registration method in accordance with an aspect of the present invention has several advantages over existing methods.

In order to generate the labeled segmentation for registration purpose, only one acquisition of MR data with two UTE volumes are required, compared to at least three sequential acquisitions of MR volumes required by the method described in “[4] van der Bom M J et al., “Registration of 2D x-ray images to 3D MRI by generating pseudo-CT data”, Phys Med Biol. 2011 Feb. 21; 56(4):1031-43. Epub 2011 Jan. 21.”

Bony structures are explicitly and reliably detected, which are the most important features for an accurate DRR-based registration using native X-ray images. In comparison, the method as described in “[4] van der Bom M J et al., “Registration of 2D x-ray images to 3D MRI by generating pseudo-CT data”, Phys Med Biol. 2011 Feb. 21; 56(4):1031-43. Epub 2011 Jan 21” (“van der Bom”) does not explicitly detect the bony structures. When there is no signal at the cortical bone in all the acquired volumes using the standard protocols as presented in the above referred to van der Bom publication, the regression method provided therein will not be able to recover the cortical bone. This can lead to a wrong registration in van der Bom, for instance to a wrong scaling in 2D projection that is then usually mapped to the wrong depth estimation in 3D.

The dual-echo UTE data sets will intrinsically register to each other so that no extra step is needed to register the MR data, in contrast to the sequential acquisition provided in the van der Bom publication.

UTE technique as provided herein may be potentially faster than separate sequential acquisitions, since the different echoes are acquired within about 10-15 ms of each other at most and as close as 2 ms for each k-space line.

Standard DRR-based 2D3D registration methods can be readily applied to align the MR volume by using the DRRs generated from the labeled segmentation from dual-echo UTE datasets, as provided herein in accordance with an aspect of the present invention. The standard techniques for DRR generation cast rays using a known camera geometry through the 3D volume, and the DRR pixel values are simply the summation of the values of those volume voxels encountered along each projection ray. The standard ray casting algorith runs in time O(n³) and hence is computationally expensive. O(n³ m) refers to computational complexity wherein n is approximately the size (in voxels) of one side of the DRR as well as one side of the 3-D volume. Further description can be found in “[8]. Fast calculation of digitally reconstructed radiographs using light fields, Daniel B. Russakoff, Torsten Rohlfing, Daniel Rueckert, Ramin Shahidi, Daniel Kim, Calvin R. Maurer, Jr., Proc. SPIE 5032, 684 (2003)” which is incorporated herein by reference.

Various fast versions of DRR generation based on GPU acceleration such as light field rendering are known.

In accordance with an aspect of the present invention the DRR is optimized and sped-up by utilizing the segmentation. In accordance with an aspect of the present invention optimization is achieved by generating a mesh representation from the segmentation, calculating intersections between a ray and the mesh triangles and then calculating the distance between the in and out intersection points on each ray. This can be accelerated by utilizing the list of intersection points between a ray and the mesh model that are provided by various ray tracing acceleration structures, such as the Octree, and GPU-assisted ray tracing.

In accordance with a further aspect of the present invention atlas information is incorporated into the bone classifier for reliable bone identification.

In accordance with an aspect of the present invention, other MR imaging protocols, such as Dixon imaging for water/fat visualization is used for generating segmentations that label different organs/tissues.

The methods as provided herein are, in one embodiment of the present invention, implemented on a system or a computer device. A system illustrated in FIG. 8 and as provided herein is enabled for receiving, processing and generating data. The system is provided with data that can be stored on a memory 1801. Data may be obtained from a medical imaging machine such as an MR machine or X-ray images or may be provided from any other data relevant source. Data may be provided on an input 1806. Such data may be image data. The processor is also provided or programmed with an instruction set or program executing the methods of the present invention that is stored on a memory 1802 and is provided to the processor 1803, which executes the instructions of 1802 to process the data from 1801. The processor 1803 can and does implement all of the previously described steps. Data, such as image data or any other data provided by the processor can be outputted on an output device 1804, which may be a computer display to display generated images such 2D3D aligned images or a data storage device. The output device 1804 in one embodiment of the present invention is a screen or display, where upon the processor displays an image which is generated in accordance with one or more of the methods provided as an aspect of the present invention. The processor also has a communication channel 1807 to receive external data from a communication device and to transmit data to an external device. The system in one embodiment of the present invention has an input device 1805, which may include a keyboard, a mouse, a pointing device, or any other device that can generate signals that represent data to be provided to processor 1803.

The processor can be dedicated hardware. However, the processor can also be a CPU or any other computing device that can execute the instructions of 1802. Accordingly, the system as illustrated in FIG. 8 provides a system for processing of image data resulting from a medical imaging device or any other data source and is enabled to execute the steps of the methods as provided herein as an aspect of the present invention.

A patient herein is any human or animal undergoing a scan or illumination by a medical imaging device, including MR, CT and X-ray device. A patient herein is thus a subject for imaging or scanning and is not required to have an illness.

Thus, systems and methods for 2D3D registration for MR-X-ray fusion utilizing one acquisition of MR data have been provided and described herein.

The following references provide background information generally related to the present invention and are hereby incorporated by reference: [1] R. Liao, C. Guetter, C. Xu, Y. Sun A. Khamene, F. Sauer, “Learning-Based 2D/3D Rigid Registration Using Jensen-Shannon Divergence for Image-Guided Surgery”, MIAR '06; [2] R. Liao, “Registration Of Computed Tomographic Volumes With Fluoroscopic Images By Spines For EP Applications”, ISBI '10; [3] James G. Reisman and Christophe Chefd'hotel “A Method for Using Ultra-short Echo Time MR to Generate Pseudo-CT Image Volumes for the Head”, Provisional Patent Application Ser. No. 61/346,508 filed May 20, 2010; [4] van der Bom M J et al., “Registration of 2D x-ray images to 3D MRI by generating pseudo-CT data”, Phys Med Biol. 2011 Feb. 21; 56(4): 1031-43. Epub 2011 Jan. 21; [5] Y. Freund and R. E. Schapire, A decision-theoretic generalization of on-line learning and an application to boosting. J. Comput. Syst. Sci. 55(1): 119-139, 1997 [6] Bergin C J, Pauly J M, Macovski A. “Lung parenchyma: projection reconstruction MR imaging”, Radiology. 1991 June; 178(2):777-81; and [7] Robson M D. Bydder G M, “Clinical ultrashort echo time imaging of bone and other connective tissues”, NMR in Biomedicine. 2006 November; 19(7):765-80; [8] Daniel B, Russakoff et al., Fast calculation of digitally reconstructed radiographs using light fields, Proc. SPIE 5032, 684 (2003); and [9] U.S. Patent Application Publication Ser. No. 20110286649 to Reisman et al. published on Nov. 24, 2011.

While there have been shown, described and pointed out fundamental novel features of the invention as applied to preferred embodiments thereof, it will be understood that various omissions and substitutions and changes in the form and details of the methods and systems illustrated and in its operation may be made by those skilled in the art without departing from the spirit of the invention. It is the intention, therefore, to be limited only as indicated by the scope of the claims.

1. A method for aligning a two-dimensional (2D) X-ray image of a patient with a Magnetic Resonance (MR) volume, comprising:
creating data representing a bony structure classifier from three-dimensional (3D) image data generated from a plurality of individuals;
acquiring with a Magnetic Resonance Imaging (MRI) device from the patient a dual echo signal volume containing an ultra-short echo time (UTE1) volume and a standard echo time (UTE2) volume;
a processor generating a labeled segmentation of the bony structure of the patient by using data representing the UTE1 and UTE2 volumes and the bony structure classifier;
the processor generating a digitally reconstructed radiograph (DRR) image from the labeled segmentation of the bony structure; and
the processor registering the DRR image with the 2D X-ray image of the patient.

2. The method of claim 1, wherein the MR volume of the patient is aligned with the 2D X-ray image.

3. The method of claim 1, wherein the DRR image is generated by the processor from the labeled segmentation by using corresponding Hounsfield Units.

4. The method of claim 1, wherein the DRR is generated by using ray-casting through the acquired MR volume.

5. The method of claim 1, wherein the DRR is generated by using GPU-based acceleration.

6. The method of claim 1, wherein the DRR is generated by using ray-casting through the acquired MR volume and GPU-based acceleration.

7. The method of claim 1 wherein the bony structure is cortical bone.

8. The method of claim 1, further comprising:
the processor generating a mesh of mesh triangles representing the labeled segmentation;
the processor calculating an intersection of a ray and a mesh triangle; and
the processor calculating a distance between an in intersection and an out intersection of the ray.

9. The method of claim 1, wherein the labeled segmentation includes a label air, a label fat or soft tissue and a label bone.

10. The method of claim 1, wherein atlas information is incorporated into the bony structure classifier.

11. A system to align a two-dimensional (2D) X-ray image of a patient with a Magnetic Resonance (MR) volume, comprising:
a memory enabled to store data;
a processor enabled to execute instructions to perform the steps:
receiving data representing a bony structure classifier from three-dimensional (3D) image data generated from a plurality of individuals;
receiving data acquired with a Magnetic Resonance Imaging (MRI) device from the patient representing a dual echo signal volume containing an ultra-short echo time (UTE1) volume and a standard echo time (UTE2) volume;
generating a labeled segmentation of the bony structure of the patient by using data representing the UTE1 and UTE2 volumes and the bony structure classifier;
generating a digitally reconstructed radiograph (DRR) image from the labeled segmentation of the bony structure; and
registering the DRR image with the 2D X-ray image of the patient.

12. The system of claim 11, wherein the MR volume of the patient is aligned with the 2D X-ray image.

13. The system of claim 11, wherein the DRR image is generated by the processor from the labeled segmentation by using corresponding Hounsfield Units.

14. The system of claim 11, wherein the DRR is generated by using ray-casting through the acquired MR volume.

15. The system of claim 11, wherein the DRR is generated by using GPU-based acceleration.

16. The system of claim 11, wherein the DRR is generated by using ray-casting through the acquired MR volume and GPU-based acceleration.

17. The system of claim 11, wherein the bony structure is cortical bone.

18. The system of claim 11, further comprising:
generating a mesh of mesh triangles representing the labeled segmentation;
calculating an intersection of a ray and a mesh triangle; and
calculating a distance between an in intersection and an out intersection of the ray.

19. The system of claim 11, wherein the labeled segmentation includes a label air, a label fat or soft tissue and a label bone.

20. The system of claim 11, wherein atlas information is incorporated into the bony structure classifier.
